SEQUENCE LISTING (14 sequences)

SEQ ID NO: 1 - protein, 146 aa, Canis sp.
MKFLVLAALLTVAAAEGGISPRAVWQFRNMIKCTIPESDPLKDYNDYGCYCGLGGSGTPVDELDKCCQTHDHCYSEAKKLDSCKFLLDNPYTKIYSYSCSGSEITCSSKNKDCQAFICNCDRSAAICFSKAPYNKEHKNLDTKKYC

SEQ ID NO: 2 - protein, 138 aa, Trimeresurus flavoviridis
MRTLWIMAVLLVGVDGGLWQFENMIIKVVKKSGILSYSAYGCYCGWGGRGKPKDATDRCCFVHDCCYGKVTGCNPKLGKYTYSWNNGDIVCEGDGPCKEVCECDRAAAICFRDNLDTYDRNKYWRYPASNCQEDSEPC

SEQ ID NO: 3 - protein, 148 aa, Homo sapiens
MKLLVLAVLLTVAAADSGISPRAVWQFRKMIKCVIPGSDPFLEYNNYGCYCGLGGSGTPVDELDKCCQTHDNCYDQAKKLDSCKFLLDNPYTHTYSYSCSGSAITCSSKNKECEAFICNCDRNAAICFSKAPYNKAHKNLDTKKYCQS

SEQ ID NO: 4 - protein, 145 aa, Notechis scutatus
MYPAHLLVLLTVCVSLLEASSIPARPLNLYQFGNMIQCANHGRRPTLAYADYGCYCGAGGSGTPVDELDRCCKAHDDCYGEAGKKGCYPTLTLYSWQCIEKTPTCNSKTGCERSVCDCDATAAKCFAKAPYNKKNYNIDTEKRCQ

SEQ ID NO: 5 - protein, 145 aa, Bungarus multicinctus
MNPAHLLILSAVCVSLLGAANVPPQHLNLYQFKNMIVCAGTRPWIGYVNYGCYCGAGGSGTPVDELDRCCYVHDNCYGEAEKIPGCNPKTKTYSYTCTKPNLTCTDAAGTCARIVCDCDRTAAICFAAAPYNINNFMISSSTHCQ

SEQ ID NO: 6 - protein, 138 aa, Vipera ammodytes
MRTLWIVAVCLIGVEGSLLEFGMMILGETGKNPLTSYSFYGCYCGVGGKGTPKDATDRCCFVHDCCYGNLPDCSPKTDRYKYHRENGAIVCGKGTSCENRICECDRAAAICFRKNLKTYNYIYRNYPDFLCKKESEKC

SEQ ID NO: 7 - protein, 138 aa, Bothrops jararacussu
MRTLWIMAVLLVGVEGDLWQFGQMILKETGKLPFPYYTTYGCYCGWGGQGQPKDATDRCCFVHDCCYGKLTNCKPKTDRYSYSRENGVIICGEGTPCEKQICECDKAAAVCFRENLRTYKKRYMAYPDVLCKKPAEKC

SEQ ID NO: 8 - protein, 145 aa, Bos taurus
MRLLVLAALLTVGAGQAGLNSRALWQFNGMIKCKIPSSEPLLDFNNYGCYCGLGGSGTPVDDLDRCCQTHDNCYKQAKKLDSCKVLVDNPYTNNYSYSCSNNEITCSSENNACEAFICNCDRNAAICFSKVPYNKEHKNLDKKKC

SEQ ID NO: 9 - protein, 145 aa, Laticauda laticaudata
MYPAHLLLLLAVCVSLLGASAIPPLPLNLAQFALVIKCADKGKRPRWHYMDYGCYCGPGGSGTPVDELDRCCKTHDQCYAQAEKKGCYPKLTMYSYYCGGDGPYCNSKTECQRFVCDCDVRAADCFARYPYNNKNYNINTSKRCK

SEQ ID NO: 10 - protein, 30 aa, elm seeds
  Xaa at positions 1, 23, 24 and 25 can be any amino acid.
XNVGVQATGTSISVGKGCKRKCXXXFCYGP

SEQ ID NO: 11 - protein, 83 aa, rice green shoots
  Xaa at position 81 can be any amino acid.
MRFFLKLAPRCSVLLLLLLVTASRGLNIGDLLGSTPAKDQGCSRTCESQFCTIAPLLRYGKYCGILYSGCPGERPCDALDXCC

SEQ ID NO: 12 - protein, 88 aa, rice green shoots
  Xaa at positions 79 and 82 can be any amino acid.
MRFFLKLAPRCSVLLLLLLVTASRGLNIGDLLGSTPAKDQGCSRTCESQFCTIAPLLRYGKYCGILYSGCPGERPCDGXDGXCMVHDH

SEQ ID NO: 13 - protein, 138 aa, rice green shoots
MPPRSPLLALVFLAAGVLSSATSPPPPPCSRSCAALNCDSVGIRYGKYCGVGWSGCDGEEPCDDLDACCRDHDHCVDKKGLMSVKCHEKFKNCMRKVKKAGKIGFSRKCPYEMAMATMTSGMDMAIMLSQLGTQKLEL

SEQ ID NO: 14 - protein, 35 aa, Ulmus glabra (seeds of elm)
  Xaa at positions 1 and 31 can be any amino acid; Xaa at position 19 is Phe or Ser; at position 23 Glu or Pro; at position 24 Pro or Lys; at position 25 Phe, Tyr or Leu; at position 34 Arg or Leu; and at position 35 Tyr or Ser.
XNVGVQATGTSISVGKGCXRKCXXXFCYGPXFLXX

(19) United States
(12) Patent Application Publication
(10) Pub. No.: US 2004/0073973 A1
(43) Pub. Date: Apr. 15, 2004

(54) Plant enzyme and use thereof

(76) Inventors: Sten Stymne, Svalov (SE); Ulf Stahl, Uppsalla (SE); Bo Ek, Bjorklinge (SE); Staffan Sjodahl, Uppsala (SE)

Correspondence Address: BROWDY AND NEIMARK, P.L.L.C., 624 NINTH STREET, NW, SUITE 300, WASHINGTON, DC, 20001-5303, US

(21) Appl. No.: 09/917,805

(22) Filed: Jul. 31, 2001

Related U.S. Application Data
(63) Continuation of application No. 09/155,124, filed on Mar. 2, 1999, now abandoned.
Application No. 09/155,124 is a 371 of international application No. PCT/SE97/00554, filed on Mar. 27, 1997.

(30) Foreign Application Priority Data
Mar. 29, 1996 (SE) 9601237.2

(51) Int. Cl. A01H 1/00; C12N 15/82; C12P 7/64; C12N 1/16; C12N 1/18

(52) U.S. Cl. 800/281; 435/134; 435/254.3; 435/254.2

(57) ABSTRACT

The invention relates to a method of identifying herbicides and to the use of inhibitors of plant peptide deformylase as broad spectrum herbicides.

### TECHNICAL FIELD

The present invention relates to use of a plant enzyme gene for transformation. More specifically, the invention relates to use of a previously not described phospholipid acyl hydrolase gene in combination with a gene for an uncommon fatty acid for obtaining transgenic plants comprising both said genes.

### BACKGROUND OF THE INVENTION

There is considerable interest world-wide in producing chemical feedstocks, such as fatty acids, for industrial use from renewable plant resources rather than non-renewable petrochemicals. This concept has broad appeal to manufactures and consumers on the basis of resource conservation and provides significant opportunity to develop new industrial crops for agriculture.

There is a diverse array of unusual fatty acids in oils from wild plant species and these have been well characterized (see e.g. Badami & Patil, 1981). Many of these acids have industrial potential and this has led to interest in domesticating relevant plant species to enable agricultural production of particular fatty acids.

Development in genetic engineering technologies combined with greater understanding of the biosynthesis of unusual fatty acids, now makes it possible to transfer genes coding for key enzymes involved in the synthesis of a particular fatty acid from a wild species into domesticated oilseed crops. In this way individual fatty acids can be produced in high purity and quantities at moderate costs.

### PRIOR ART

Within prior art it is known that plant tissues accumulating triacylplycerols with high amount of medium chain (fatty acids shorter than 16 carbon atoms), hydroxy fatty acids, epoxy fatty acids and acetylenic acids have low concentration of these acids in their membrane lipids (phospholipids). (Stymne et al 1990; Bafor et al., 1990, 1991, 1993; Kohn et al., 1994).

Furthermore it is known that diacylplycerols is a common precursor for both phospholipids and triacylglycerols in plant tissues accumulating triacylglycerols (see Stymne, 1993a for review). There is also known that CDP-choline choline phosphotransferase in plant tissues accumulating high amounts of medium chain and hydroxy fatty acids in their triacylplycerols do not discriminate against diacylglycerols containing these fatty acids in the synthesis of phosphatidylcholine (Vogel & Browse, 1995).

Prior art also describes that tissues naturally accumulating triacylplycerols with medium chain fatty acids, epoxygenated fatty acids and hydroxylated fatty acids have membrane associated acyl hydrolase activities with high specifities towards phospholipids containing the particular uncommon fatty acid this tissue is accumulating, but low activity for common membrane fatty acids (Stymne, 1993, Stahl et al., 1995).

Furthermore, prior art describes that rape seed genetically engineered to produce dodecanoic (lauric) acid in their seeds have much higher content of that acid in seed phospholipids than two plant species naturally accumulating lauric acids to approximately the same relative level (Wiberg et al, 1995).

Finally, there exists prior art concerning an anonymous expressed cDNA sequences from young shoots of rice (ID's: D49050, D47724, D47653, D47320) deposited by the Rice genome Research Program in the GenBank.

### SUMMARY OF THE INVENTION

Many of the unusual fatty acids of interest, e.g. medium chain fatty acids, hydroxy fatty acids, epoxy fatty acids and acetylenic fatty acids, have physical properties that are distinctly different from the common plant fatty acids. The present inventors have found that, in plant species naturally accumulating these uncommon fatty acids in their seed oil (triacylplycerols), these acids are absent, or present in very low amounts, in the membrane (phospho) lipids of the seed. The low concentration of these acids in the membrane lipids is most likely a prerequisite for proper membrane function and thereby for proper cell functions. The idea underlying the invention is that uncommon fatty acids can be made to accumulate to high amounts in seeds of transpenic crops if these uncommon fatty acids are, more or less, excluded from the membrane lipids of the seeds.

The present invention relates to genetically engineering of oil seeds, oleogeneous yeast and moulds to accommodate high amounts of uncommon fatty acids in their triacylglycerols by introducing genes coding for phospholipid hydrolases, below also called phospholipases, that specifically removes these fatty acids from the membrane lipids of the cell.

The inventors have identified phospholipase (phospholipase A₂) enzymes responsible for the removal of medium chain fatty acids from phospholipids in plants.

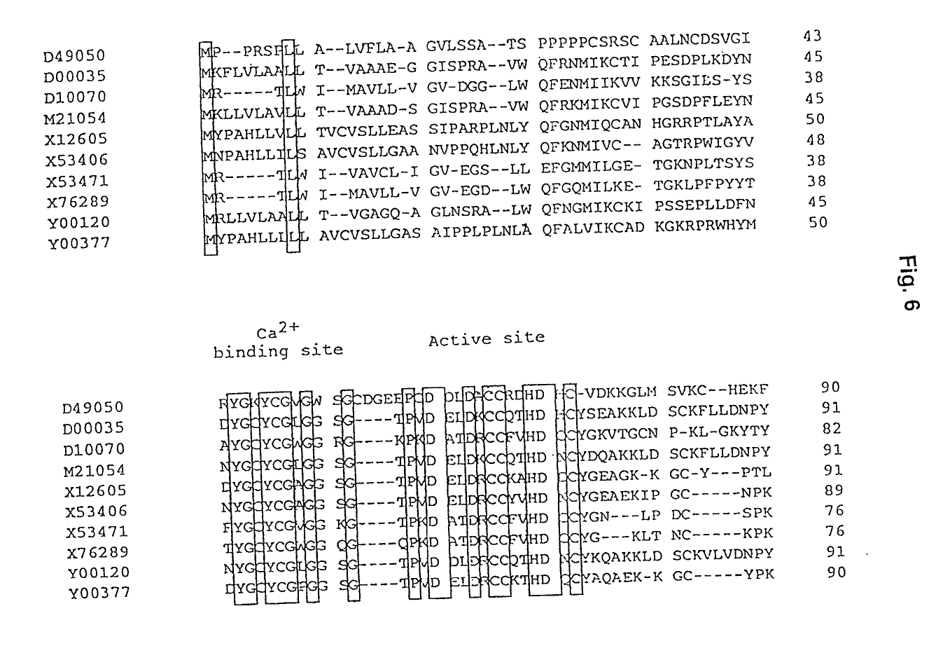

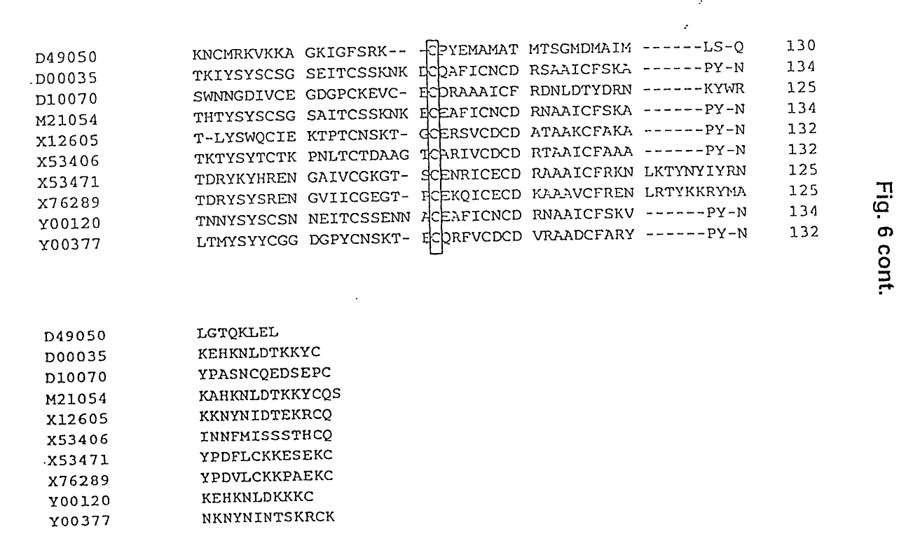

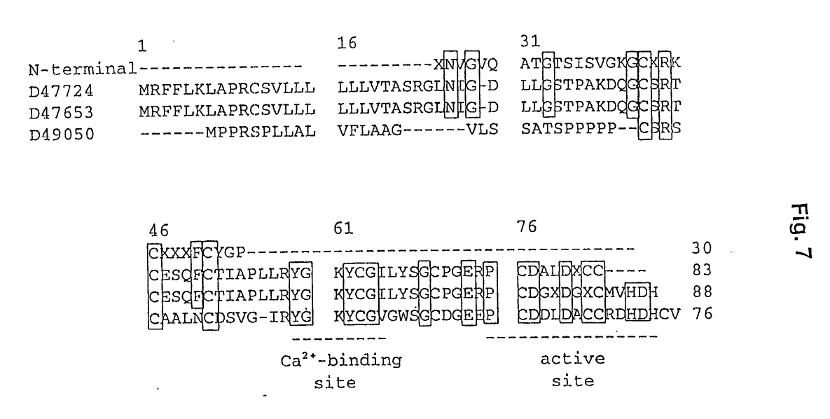

Thus, in a first aspect the present invention relates to cDNA or genomic DNA coding for a phospholipid acyl hydrolase comprising a nucleotide sequence coding for an amino acid sequence with homology to Ulmus glabra phospholipase A₂ as presented in FIG. 7 or amino acid sequences homologous to those encoded by the rice cDNA clones D49050, D47724, D47653 as presented in FIGS. 6 and 7.

In a second aspect, the invention relates to the use of a plant phospholipid hydrolase gene (cDNA or genomic DNA coding for a phospholipid hydrolase) in combination with a gene for an uncommon fatty acid for obtaining transgenic plants comprising both said genes.

Preferably, the enzyme encoded by said phospholipid acyl hydrolase gene, or cDNA, is coding for a low molecular weight phospholipase A₂ with distinct acyl specificity for uncommon fatty acids, such as medium chain, long chain (>C₁₈), hydroxy, epoxy and acetylenic acids.

In a third aspect, the invention relates to transgenic oil accumulating organisms comprising, in their genome, a plant phospholipid hydrolase gene having specificity for a particular uncommon fatty acid and the gene for said uncommon fatty acid.

Preferably said organisms are selected from the group consisting of oil crops, yeasts, and moulds.

In a fourth aspect, the invention also relates to oils from such organisms.

### DETAILED DESCRIPTION OF THE INVENTION

Studies by the inventors on the biosynthesis and metabolism of uncommon fatty acids (i.e. medium chain fatty acids, hydroxy fatty acids, epoxy fatty acids) in different oil seeds (Bafor et al., 1991, 1993; Banas et al., 1993, Stymne, 1993, Stahl et al., 1995), led the inventors to conclude that microsomal phospholipid acyl hydrolases (phospholipases) with specifities towards uncommon acyl groups was, at least in part, responsible for the removal of these acids from the phospholipids in the developing oil seeds.

It was also shown that the acyl specificities of the phospholipid acyl hydrolases from different plant species were correlated with the type of accumulated fatty acid in the plants.

Elm (Ulmus glabra) seed triacylplycerols are mainly composed of octanoic (8:0) and decanoic (10:0) acids, but these acids are very low in concentrations in the phospholipids of the seeds (Stahl et al., 1995). Membrane fractions (microsomal preparations) from developing Ulmus glabra seeds had high phospholipase A₂ (PLA₂) activity towards phosphatidylcholine with medium chain fatty acids in position sn-2 (octanoic, decanoic and dodecanoic (12:0) acids but very low activity towards phosphatidylcholine with octadeca-9-enoic acid (oleic acid—a common fatty acids) (Stymne, 1993, Stahl et al. 1995). Microsomal preparations from developing rape seed did not have such phospholipase A₂ activity towards medium chain fatty acids (Stahl et al. 1995).

If a gene coding for plant phospholipase A₂ with specificities for a particular uncommon fatty acids is expressed in transgenic oil producing organisms engineered to produce that uncommon fatty acids, the recombinant phospholipase A₂ will remove the uncommon fatty acids from the phospholipids of the cell and thereby prevent deleterious effects on cell metabolism caused by the presence of this acid in the membrane lipids. This invention describes how such phospholipase A₂ genes will be isolated and what uses they will have in commercial applications.

The invention will be described more closely below in association with an experimental part and the enclosed drawings.

The drawings show:

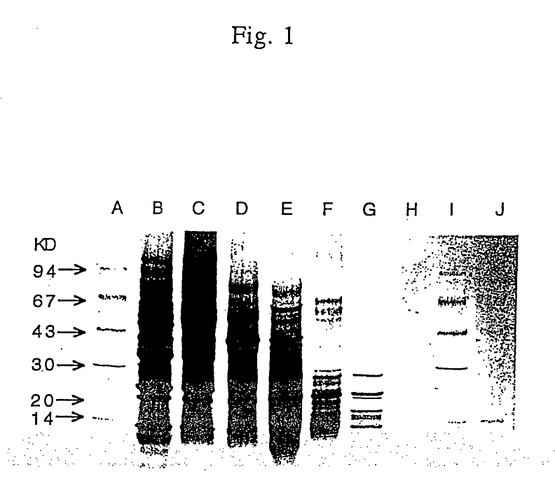

FIG. 1 shows a SDS-polyacrylamid electrophoresis of soluble developing elm seed PLA₂ purification fractions, followed by colloidal Coomassie staining. Lane A and I contain 100 ng of MW standards (Pharmacia low MW); lane B, 100,000 g supernatant, 50 μg; lane C, ammonium sulphate pellet, 50 μg; lane D, acetone supernatant, 50 μg; lane E, Q Sepharose, 40 μg; lane F, Superose 12, 25 μg; lane G, C₄—HPLC, 2 μg; lane H C₂C₁₈—SMART, 100 μg; and lane J, commercial Naja naja kouthia PLA₂, 100 ng. All samples were reduced with DTT in sample buffer.

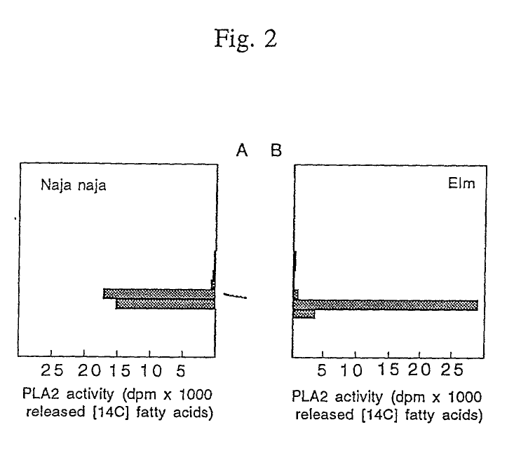

FIG. 2 shows PLA₂ activity measurements of gel pieces from whole lanes (5-94 KD) of a SDS-PAGE 8-18% gradient gel. Lane A contains 50 ng of Naja naja kouthia PLA₂ (Sigma) and lane B 50 ng of developing elm seed soluble PLA₂. PLA₂ activity recovered from gel pieces of similar lanes are shown on each side.

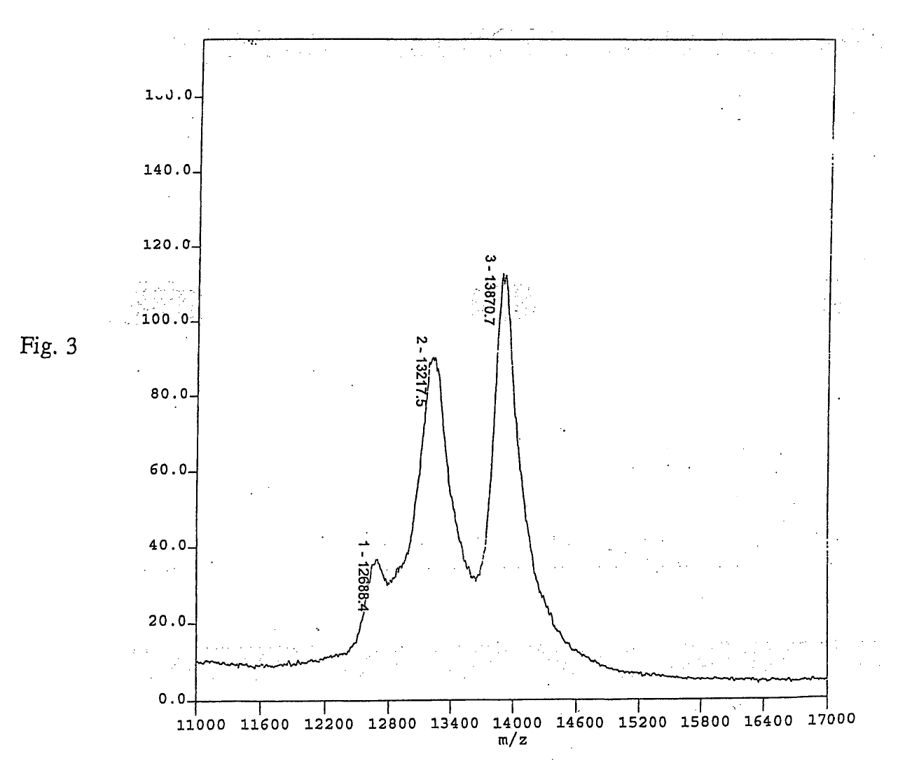

FIG. 3 shows molecular weight data of the purified soluble PLA₂, from MS-Malditof.

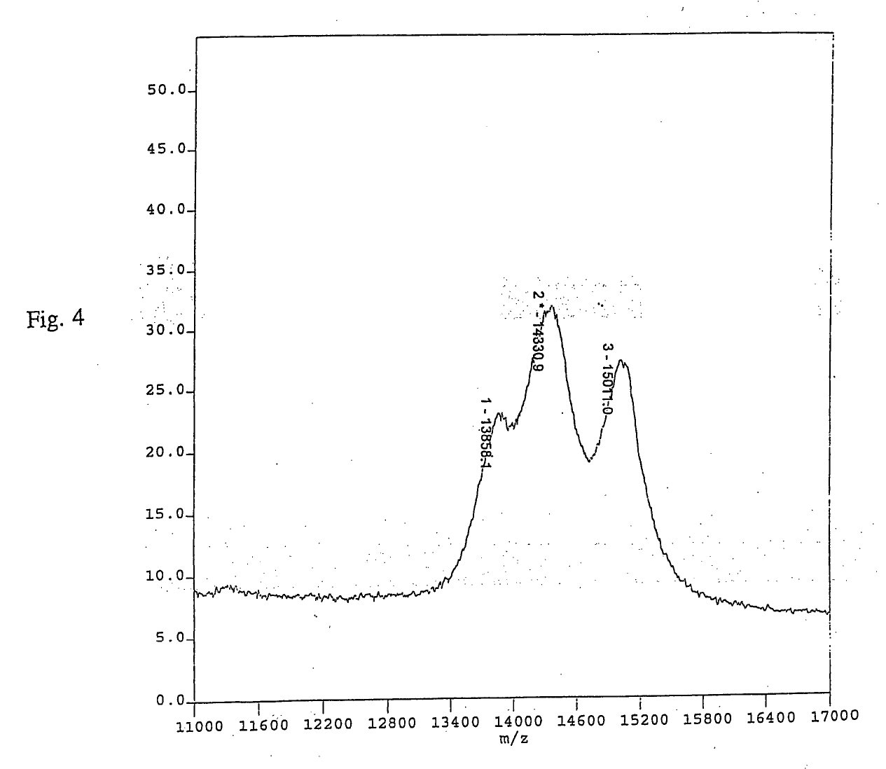

FIG. 4 shows molecular weight data of the purified soluble PLA₂ that has been reduced and iodoacetamide alkylated, from MS-Malditof.

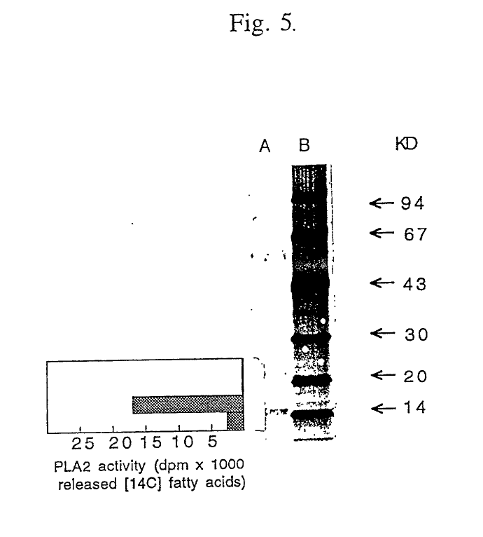

FIG. 5 shows a SDS-polyacrylamid electrophoresis of purified microsomal peak 11 PLA₂ from developing elm seeds, with recovered PLA₂ activity. Lane A contain about 20 ng of purified peak II PLA₂, non reduced; lane B contain 25 ng of MW standard (Pharmacia low MW). The gel was silver stained.

FIG. 6 Alignment of deduced amino acid sequence of the full length rice cDNA clone GenBank ID: D49050 with 10 different low molecular weight phospholipase A₂ from animal tissues. Conserved amino acid sequences are boxed. Spaces introduced to optimize alignment are indicated by a dash. The different sequences represent phospholipase A₂ from the following species:

D00035: Canis sp. (SEQ ID NO:1)

D10070: Trimeresurusflavolridis (SEQ ID NO:2)

M21054: Homo sapiens (SEQ ID NO:3)

X12605: Notechis scutatus scutatus (SEQ ID NO:4

X53406: Bungarus multicinctus (SEQ ID NO:5)

X53471: Vipera ammodytes (SEQ ID NO:6)

X76289: Bothrops jararacussu (SEQ ID NO:7)

Y00120: Bostaurus (SEQ ID NO:8)

Y00377: Laticauda laticaudata (SEQ ID NO:9)

FIG. 7 Alignment of the N-terminal sequence (SEQ ID NO:10) of the purified soluble PLA₂ from elm seeds with deduced amino acid sequences (SEQ ID NOs:11-13) from three EST-clones from rice green shoots, including the cDNA clone D49050 fully sequenced by the inventors. The EST-sequences are denoted by their GenBank accession number. Conserved amino acid positions between the elm and rice proteins as well as the regions with homology to the Ca ²⁺-binding and the active site in animal low molecular weight PLA₂'s are boxed. A fourth rice clone (GenBank ID: D47320) with high homology to the three above was found in the EST database, but excluded from the alignment due to lower quality of the DNA sequence.

### EXPERIMENTAL PART

Proteins with phospholipase A₂ (PLA₂) activities towards 1-palmitoyl-2-decanoyl-sn-glycerol-3-phosphocholine were purified from a soluble and microsomal fraction of developing elm seeds according to the following protocols:

Assays of Phospholipase A₂ Activity.

Membrane associated PLA₂ activity was assayed according to Stahl et al. (1995) using sn-1-palmitoyl-sn-2-[¹⁴C]decanoyl-sn-glycerol-3-phosphocholine as substrate. In standard assays of the solubilized microsomal activity and of the soluble activity 1-palmitoyl-2-[¹²C]palmitoyl-glycerol-sn-3-phosphocholine was used as substrate and was presented as mixed micelles with the non-ionic detergent lubrol PX, in a PC/detergent molar relation of 1:10. Samples, 0.5-10 μl, were assayed for PLA₂ activity by incubation at 30° C. for 5-30 min with 5 nmol of ¹⁴C-labelled phosphatidylcholine (10,000 dpm/nmol) in a total volume of 50 μl of 50 mM Tris/HCl, pH 8.0 containing 10 mM CaCl₂ and 0.06% (w/v) lubrol PX. The reaction was stopped by the addition of 400 μl of chloroform/methanol/acetic acid 50:50:1 followed by 150 μl of H₂O. The samples were mixed thoroughly and centrifuged 10,000 g for 1 min. Chloroform phases containing extracted lipids were passed through mini-columns of silica gel, followed by a wash with 400 μl of chloroform. The eluates from the silica columns, containing released [¹⁴C]palmitic acid, were collected and analysed by scintillation counting.

Assays of PLA₂ activity from SDS-gels was performed according to following protocol; A whole or part of a SDS-PAGE (Pharmacia Exelgel 8-18%) lain, not fixed, containing purified PLA₂ were divided in 2-3 mm wide pieces and placed in eppendorf tubes together with 400 μl of 20 mM Tris, pH 8.0 containing SDS 0.5% (w/v). The tubes were rotated end over end at 37° C. for 16 h, in order to eluate proteins from the gel pieces. Fractions were concentrated to 100 μl in a Speed-Vac concentrator (Savant) and then precipitated with ethanol/chloroform (Wessel and Flugge 1984) to remove SDS. Air dried pellets were solubilized in 150 μl of 50 mM Tris/HCl, pH 8.0 containing 10 mM CaCl₂ and 0.06% (w/v) lubrol PX and activity measurements were started by adding 5 nmol of sn-1-palmitoyl-sn-2-[¹⁴C]palmitoyl-sn-glycerol-sn-3-phosphocholine (10,000 dpm/nmol) solubilized in 50 μl of 50 mM Tris/HCl, pH 8.0 containing 10 mM CaCl₂ and 0.06% (w/v) lubrol PX. The samples were incubated at 30° C. for 2-4 h and stopped by adding 400 μl of CHCl₃/MeOH/Hac, 50:50:1.

SDS-Gel Electrophoresis

Protein fractions were if necessary concentrated in Sped-Vac and precipitated with ethanol/chloroform according to Wessel and Flugge (1984). Samples with a final volume of 20 μl in 50 mM Tris/acetat pH 7.5 with 1% (w/v) SDS, with or without 10 mM of dithiothreitol, were heated to 95° C. for 5 min, centrifugated 5 min 13,000 g and loaded on to a horizontal 8-18% gradient polyacrylamid gel (Pharmacia ExelGel SDS) with a 33 mm stacking zone and a 77 mm separating zone. The gel was chromatographed on a Pharmacia Multiphor II unit at 15° C. and stained either with colloidal Coomassie (Neuhoff et al 1988) over night or with silver staining

Material

Developing seeds of elm (Ulmus glabra) were harvested and peeled. The white endosperms were immediately frozen in liquid nitrogen and stored in −80° C.

Purification of Soluble Phospholipase A₂

60 g of liquid nitrogen frozen elm endosperm was homogenized with a Ultraturcrax® in 600 ml of ice cold 100 mM potassium phosphate buffer, pH 7.2 containing 0.33 M sucrose. The homogenate was filtered through two layers of Miracloth® and centrifuged 10,000 g for 12 min. The supernatant was filtered through one layer of Miracloth® and centrifuged a second time, at 100,000 g for 90 min. The final supernatant which contained about 80% of the total PLA₂ activity, was brought to 55% (w/v) of ammonium sulphate and left with steering at 4° C. for 1 h. Precipitated proteins were pelleted by centrifugation 10,000 g for 10 min and resuspended in 130 ml of 50 mM dietanolamin buffer pH 8.5. Ice cold acetone was added to a final concentration of 45% (v/v) and the extract was left at 4° C. for 30 min. Precipitated proteins were removed by centrifugation for 10 min at 10,000 g and the resulting supernatant was dialysed against 20 volumes of 20 mM piperidin, pH 11.0, with one change over night. The dialysed extract was applied to a Q-Sepharose Fast Flow 7 ml column (1.0×10.0 cm) equilibrated in 20 mM piperidin, pH 11.0. The column was eluted with a linear salt gradient from 100 to 500 mM NaCl in 20 mM piperidin, pH 11.0 at a flow rate of 2 ml/min. 3 ml fractions were collected and assayed for PLA₂ activity. A single broad peak of activity was eluted at a salt concentration of 200 to 300 mM NaCl. Peak fractions were pooled, concentrated on Centricon-10 to 0.6 ml and chromatographed in three separate runs on a Pharmacia Superose 12 (1.0×30.0 cm) gel filtration column (0.4 ml/min) in 20 mM Tris/HCl, 150 mM NaCl, pH 8.0. Fractions (0.5 ml) were collected and tested for PLA₂ activity. Peak fractions from all three runs were pooled and the PLA₂ was further purified using a C₄ reversed-phase HPLC column (Vydac 0.46×10.0 cm) that was equilibrated in 0.1% trifluoroacetic acid (TFA). The column was developed at 0.4 ml/min with a 30 min gradient (20-45% of acetonitrile in 0.1% TFA) and peaks monitored at 280 nm were collected manually. Collected fractions from four separate runs were assayed for PLA₂ activity. Peak fractions were pooled and the acetonitrile content was reduced by evaporation in a Speed-Vac concentrator (Savant). The PLA₂ was finally purified to apparent homogeneity on a C₂C₁₈ reversed-phase HPLC column (0.21×10.0 cm) equilibrated in 0.1% TFA and developed at 100 μl/min with a 60 min gradient (30-60% acetonitrile in 0.1% TFA) using a SMART system (Pharmacia). Peaks monitored at 280 nm were automatically collected and then subjected to PLA₂ assay. The PLA₂ elutes as a discrete peak in the gradient at about 50% acetonitrile.

The PLA₂ was purified about 180 000 times from the developing elm seed extract of soluble proteins, to a final specific activity of 44 mmol/min×mg protein (see Table I).

TABLE I
Purification of Soluble PLA₂ from Developing Elm Seeds

| | | | Specific | | |
|---|---|---|---|---|---|
| | | | Activity | | |
| | | Total | (nmol/min | | |
| | Protein | Activity | × mg | Purifi- | |
| | (mg) | (nmol/ml) | protein) | cation | Yield (%) |
| 100,000 g sup | 3,340 | 833 | 0.25 | 1 | 100 |
| Am. sulphate pell | 1,060 | 563 | 0.53 | 2 | 68 |
| Acetone sup | 150 | 780 | 5.2 | 21 | 94 |
| Q Seph F.F. | 24 | 420 | 17.5 | 70 | 50 |
| Gel Filtration | 3.8 | 263 | 69.2 | 277 | 32 |
| C₄-HPLC | 0.09 | 173 | 1,922 | 7,690 | 21 |
| C₂C₁₈-SMART | 0.003 | 133 | 44,330 | 177,300 | 16 |

The final extract showed one major band when subjected to SDS-PAGE on a 8-18% gel and stained with colloidal Coomassie, with a molecular mass of 14 kDa (see FIG. 1). Recovered PLA₂ activity from SDS-PAGE gels coincide with the 14 kDa band (see FIG. 2). When subjected to Malditof-MS, the PLA₂ gave two major peaks with the masses 13220 and 13890 and a minor with a molecular mass of 12680 (see FIG. 3). When alkylated with N-isopropyl all three peaks changed masses with about the same amount, 1150, which would correspond to 12 cysteine residues in each of the three proteins (FIG. 4).

N-Terminal Sequence Analysis

About 1 μg of purified PLA₂ was reduced, by incubation in 0.1 M Tris/HCl, pH 8.5 containing 8 M guanidinhydrocloride, 10 mM EDTA and 20 mM DTT at 560 C for 30 min followed by alkylation in 20 mM 4-vinylpyridin for 60 min at room temperature. The reduced and alkylated PLA₂ was desalted, applied on a C₂C₁₈ g reversed-phase HPLC column (0.21×10.0 cm) equilibrated in 0.1% TFA and eluted at 100 μl/min with a 30 min gradient (30-60% acetonitrile in 0.1% TFA) using a SMART system (Pharmacia). The protein was then subjected to amino-terminal sequence determination by automated Edman degradation using an Aplied Biosystems 476A gase phase protein sequenator. The amino-terminal sequence was manually determined to be: XNVGVQATGTSISVGKGCF(S)RKCE(P)P(K)F(Y,L)FCYGPXFLR(L)Y(S) (SEQ ID NO:14) (when signals for several amino acids were obtained the minor amino acid signal(s) is denoted in brackets after the main signal). When using the amino-terminal sequence as query for the Basic local alignment search tool at NCBI with the blastp search program against the Non-redundant GenBank CDS translations+PDB+SwissProt+SPupdate+PIR, the tblastn search program against Non-redundant GenBank+EMBL+DDBJ+PDB sequences and Non-redundant Database of GenBank EST Division the best aligned sequences are three EST's (GenBank accession number D47724, D47653 and D47320) derived from green rice shoots. FIG. 7 shows an alignment of the amino-terminal sequence with the deduced amino-acid sequence from two of these EST-clones and the D49050 rice EST-clone. The amino-terminal sequence show significant homology with the rice sequences, notably the positions of the three cysteine-residues are conserved. In addition, predictions of leader peptide cleavage site of rice clones D47724 and D47653 suggest cleavage between G 24 and L 25. This supports the alignment of the amino-terminal sequence to the mature part of the rice sequences Regions with high homology to the conserved Ca²⁺ binding- and active sites of secretory PLA₂'s (see FIG. 6) are both found in all three aligned rice EST's.

Purification of Microsomal Phospholipase A₂

60 g of liquid nitrogen frozen elm endosperm was homogenized with a ultraturrax® in 600 ml of ice cold 100 mM potassium phosphate buffer, pH 7.2 containing 0.33 M sucrose. The homogenate was filtered through two layers of Miracloth® and centrifugated 10,000 g for 12 min. The supernatant was filtered through one layer of Miracloth® and centrifugated a second time, at 100,000 g for 90 Min. The microsomal pellets were resuspended in 90 ml of 100 mM potasium phosphate, pH 7.2 with a glass homogenizer and, if not used immediately, stored at −80° C. The microsomal membranes were diluted to 150 ml with 100 mM potasium phosphate, pH 7.2 and solubilized by the addition of 150 ml of 200 mM potassium phosphate, pH 7.2 containing glycerol 17% (v/v), lubrol PX 0.6% (w/v) and EGTA 1 mM. The mixture was incubated 15 min at 4° C. followed by a centrifugation 100 000 g at 40 C for 90 min. The supernatant was dialysed against two changes of 5 liter of 20 mM dietanolamin, pH 8.5 containing glycerol 8.7% (v/v) and lubrol PX 0.06% (w/v). The dialysed supernatant was passed through a 200 ml Q-Sepharose column (50×100 mm) at a flow rate of 3 ml/min. Non-retained material with about 30-50% of the PLA₂ activity was collected and pH adjusted to 5.7 by adding requiring amount of 1 M MES buffer. This fraction was applied to a 7 ml SP-Sepharose column (10×100 mm) equilibrated in 50 mM MES, pH 5.7 with glycerol 8.7% (v/v) and lubrol PX 0.06%(w/v) at a flow rate of 3 ml/min. After the sample had passed through, the column was washed with several column volumes of equilibrating buffer and then eluted with a 100 ml linear gradient from 0 to 480 mM of NaCl in the same buffer. Eluated fractions containing PLA₂ activity were pooled and concentrated to a volume of 200 μl by centrifugation on Centricon-50 and vacuum evaporation in a Speed-Vac concentrator (Savant). The sample was applied to a Superose 12 (10×300 mm) Pharmacia column equilibrated in 20 mM Tris, pH 8.0 with glycerol 4.3% (v/v), lubrol PX 0.06% (w/v) and 50 mM NaCl. Fractions with PLA₂ activity were pooled and further purified using a C₄ reversed-phase HPLC column (Vydac 0.46×10.0 cm) that was equilibrated in 0.1% trifluoroacetic acid (TFA). The column was developed at 0.4 ml/min with a 30 min gradient (20-45% of acetonitrile in 0.1% TFA) and peaks monitored at 280 nm were collected manually. Collected fractions were assayed for PLA₂ activity, and found to be divided into to activity peaks, one (peak I) which eluted at about 35% acetonitrile and the second (peak II) which eluted at about 47% acetonitrile. Peak fractions were pooled and lubrol PX to a final concentration of 0.5% (w/v) was added before the acetonitrile content was reduced by evaporation in a Speed-Vac concentrator (Savant). The two PLA₂ fractions were both finally purified to near homogeneity on a C₂C₁₈ reversed-phase HPLC column (0.21×10.0 cm) equilibrated in 0.1% TFA and developed at 30 μl/min with a 60 min gradient (30-60% acetonitrile in 0.1% TFA) using a SMART system (Pharmacia). Peaks monitored at 280 nm were automatically collected and then subjected to PLA₂ assay. The purified peak I PLA₂ gave a very sharp band on SDS-PAGE 8-18% gradient gel with a molecular mass around 17 KD and the peak II PLA₂ gave a 14 KD band. Both bands coincided with recovered PLA₂ activity from gel pieces. FIG. 5 shows the purified peak 11 PLA₂ separated on a SDS-PAGE followed by silverstaining. After Coomassie staining only the 14 KD band was visible. However, upon silverstaining some minor contaminants show up.

The microsomal PLA₂ activity was purified from the microsomal fraction with a specific activity of 0.28 nmol/min×mg protein to a specific activity of about 50 mmol/min×mg protein which gives a purification factor of about 100,000.

Properties of Purified PLA₂s

The purified soluble and microsomal PLA₂s have very similar properties. They have a pH optimum between 7 and 9, an absolute requirement for Ca²⁺ for activity with several mM for optimal activity. The activities are extremely stable both to extreme pH values, heat and organic solvents. The activities are, however, sensitive to reducing agents like DTT and mercaptoethanol (see Table II).

TABLE II
Effects of Reduction, EGTA and Heat on Developing
Elm Seed Soluble PLA₂

| | PLA₂ Activity Released |
|---|---|
| Treatment | [¹⁴C] fatty acids (dpm) |
| Control | 4740 |
| 05° C for 5 min | 5190 |
| Mercaptoethanol 1% (v/v) | 170 |
| EGTA 10 mM | 170 |

The purified PLA₂'s hydrolyses the sn-2 position of phospholipids (Table III), and does not show any activity towards diacylplycerols or lysophosphatidylcholine (Table IV).

TABLE III
Position specificity of soluble developing elm seed
PLA₂ assay described above with a partly
purified soluble PLA₂ fraction (PC =
phosphatidylcholine, LPC = lysophosphatidyl-
choline)

Recovered ¹⁴C Activity (% of total recovery)

| PC Substrate | fatty acid | PC | LPC |
|---|---|---|---|
| sn-1(16:0-sn- | 44 | 56 | 0.6 |
| 2-[¹⁴C]16:0 | | | |
| di-[¹⁴ C]16:0 | 27 | 50 | 23 |

TABLE IV
Substrate specificity of microsomal PLA₂.
Incubations done according to the PLA₂ assay
described above (PC = phosphatidylcholine,
LPC = sn-1-lysophosphatidylcholine,
DAG = Diacylglycerol)

Recovered ¹⁴C activity
(% of total recovery)

| | | | | |
|---|---|---|---|---|
| PC Substrate | fatty acid | PC | LPC | DAG |

| | | | | |
|---|---|---|---|---|
| sn-1-16:0- | 34 | 65 | 0.8 | — |
| sn-2-[¹⁴C]16: | | | | |
| 0-PC | | | | |
| di-[¹⁴C]16:0- | 27 | 40 | 33 | — |
| PC | | | | |
| sn-1-16:0- | 44 | 54 | 0.6 | |
| sn-2-[¹⁴C]10: | | | | |
| 0-PC | | | | |
| [¹⁴C]10:0-LPC | 0.8 | — | 98 | — |
| sn-1-16:0- | 0.8 | — | — | 99 |
| sn-2-[¹⁴C]10: | | | | |
| 0-DAG | | | | |

The molecular weight and the biochemical characteristics of both the soluble and microsomal elm PLA₂ suggest that they are related to the well described low MW “secretory” PLA₂s from animal sources. This is further supported by the amino-terminal sequence data and alignments. The secretory PLA₂s have all conserved amino acid sequences at the Ca²⁺ binding site and at the active site as well as cyein residues.

When searching databases for deposited expressed sequences from plants with homology to low molecular weight animal phospholipases in Ca²⁺ binding site and active site, the inventors found three anonymous partially sequenced cDNA clones from green shots of (GenBank ID: D49050, D47724, D47653). The cDNA clone D49050 was received upon request from Dr. Yoshiaka Nagamura, DNA Materials Management group, Rice Genome Project, NIAR/STAFF, STAFF Institute, 446-1, Ippaizuka, Kamiyokoba Tsukuba, Ibaraki 305 Japan. The entire cDNA was sequenced and was shown to contain an open reading frame encoding a full length protein of an estimated molecular weight of 15 kDa. An alignment of the deduced amino acid sequence of D49050 with a number of animal low molecular weight PLA₂s is presented in FIG. 6.

The D49050, D47724, D47653 clones coded for proteins with the same amino acid sequences as in thee Ca²⁺ binding site and active site in the animal low molecular weight PLA₂s and similar to these enzymes they contained several cysteine residues (see FIG. 7). The cDNA clones also coded for amino acid sequences with significant homologies with the N-terminal sequence of the purified phospholipase A₂ from elm seeds where the positions of the three cysteine residues of the elm enzyme was totally conserved in all three cDNAs (see FIG. 7). Thus with all probability these rice cDNAs were coding for a plant PLA₂ similar to the enzyme purified from developing elm seeds according to the invention.

By expressing this cDNA in suitable organism, like bacteria, for example E. coli, yeast or plants, a recombinant PLA₂ protein will be obtained and PLA₂ activities can be demonstrated. Although the physiological function of the rice enzyme is unknown, a function in rice shoots could be removal of oxygenated fatty acids from membrane lipids, as has been shown to take place in e.g. wheat roots (Banes et al, 1992).

By constructing degenerated nucleotide primers based on suitable amino acid sequences of the soluble elm PLA₂ and rice cDNA clones amplification of elm fragments containing the corresponding sequences will be done from cDNA or genomic DNA from elm seeds by PCR. These fragments will be used as probes for screening for the elm cDNA PLA₂ from a cDNA library from developing elm seeds.

Since phospholipid acyl hydrolases with high specificities towards epoxy and hydroxy fatty acids have been described in membrane preparations from other plant species (Stahl et al. 1995) homologous cDNA coding for PLA₂ with other acyl specificities than the elm enzyme can be isolated from other plant species with the aid of the cDNA encoding for the elm PLA₂ and/or the rice cDNA clones as probes. Alternatively suitable amino acid sequences of these enzymes can be used to construct degenerated nucleotide primers and amplify cDNA fragments derived from the other plant species. These fragments will be used as probes for screening for the cDNA coding for PLA₂ from a cDNA library from other plant species.

When a cDNA clone containing a full length cDNA or genomic DNA coding for a PLA₂ have been obtained this cDNA can be used for transformation.

According to the invention, the PLA₂ gene, i.e. the PLA₂ cDNA or genomic clone, is used in combination with a gene for an uncommon fatty acid for obtaining transgenic plants comprising both said genes. The transgenic plants are obtained by using said plant phospholipid acyl hydrolase gene for transforming transgenic oil accumulating organisms engineered to produce said uncommon fatty acid. Alternatively, transgenic plants are obtained by using the plant phospholipid acyl hydrolase gene for transforming oil accumulating organisms, which are crossed with other oil accumulating organisms engineered to produce said uncommon fatty acid.

### REFERENCES

Badami, R. C., and Patil, K. B. (1981). Structure and occurrence of unusual fatty acids in minor seed oils. Progress in Lipid Research, 19, 119-53.

Bafor, M., Smith, M. A., Jonsson, L., Stobart, K. & Stymne, S (1990) Regulation of triacylplycerol biosynthesis in embryos and microsomal fractions from the developing seeds of Cuphea lanceolata. Biochem. J. 272, 31-38

Bafor, M., Srnitl;l, M. A., Jonsson, L., Stobart, K. & Stymne, S. (1993) Biosynthesis of vernoleate (cis-12-epoxyoctadeca-cis-9-enoate) in microsomal preparations from developing endosperm of Euphorbia lagascae. Arch. Biochem. Biophys. 303, 145-151

Banas, A., Johansson, I. & Stymne, S. (1992) Plant microsomal phospholipases exhibit preference for phosphatidylcholine with oxygenated acyl groups. Plant Science 84, 137-144

Kohn, G., Hartmann, E., Stymne, S. & Beutelmann, P. (1994) Biosynthesis of acetylenic acids in the moss Ceratodon purpureus. J. Plant Physiol. 144, 265-271

Neuhoff, V., Arold, N., Taube, D. and Ehrhart, W. (1988) Improved staining in polyacrylamide gels including isoelectric focusing gels with clear background at nanogram sensitivity using Coomassie Brilliant Blue G-250 and R-250. Electrophoresis 9, 255-262

Stymne, S (1993a) Biosynthesis of storage fat in oil crops—today and tomorrow. In: Phytochemistry and Agriculture (Eds. T. A. van Beek & H. Breteler. pp. 288-312. Clarendon Press, Oxford)

Stymne, S. (1993b) Biosynthesis of uncommon fatty acids and their incorporation into triacylglycerols. In: Biochemistry and Molecular Biology of membrane and Storage Lipids of Plants (eds. N. Murata & C. Somerville), pp. 150-158. American Society of Plant Physiology, Rockville.

Stymne, S., Bafor, M., Jonsson, L., Wiberg, E., and Stobart, A. K. (1990) Triacylglycerol assembly. In Plant Lipid Biochemistry, Structure and Utilization, (eds. P. J. Quinn and J. L. Harwood), pp. 191-97. Portland Press, London

Stahl, U., Banas, A. & Stymne, S. (1995) Plant microsomal phospholipid acyl hydrolases have selectivities for uncommon fatty acids. Plant Physiol. 107, 953-962

Voge!, G. & Browse, J. (1995) Role of choline phosphotransferase and diacylglycerol acyltransferase in channelling unusual fatty acids into the triacylglycerol pool during oil seed development. In: Plant lipid metabolism (eds. J-C. Kader & P. Mazliak), pp. 506-508. Klower Academic Press, Dordrecht

Wessel, D. and Flugge, U. I. (1984) A method for quantitative recovery of protein in diluted solution in the presence of detergents and lipids. Biochemistry 138, 141-143

Wiberg, E., Banas, A. & Stymne, S. (1995) Partitioning of medium chain fatty acids between membrane and storage lipids in laureate producing rape. Abstract 039 in abstract book from the symposium: Biochemistry and Molecular Biology of Plant Fatty acid and Glycerolipid. June 1-4, South Lake Tahoe, Calif.

What is claimed is:

1. A DNA sequence coding for a low molecular weight phospholipase A₂ with distinct acyl specific for uncommon fatty acids, comprising a nucleotide sequence coding for an amino acid sequence with essential homology to Ulmus glabra phospholipase A₂ as presented in FIG. 7 or amino acid sequences essentially homologous to those encoded by the rice cDNA clones D49050, D47724, D47653 as presented in FIGS. 6 and 7.

2. A method of accumulating uncommon fatty acids. in the triacylglycerols of oil seeds, oleogeneous yeasts and moulds; comprising removing said uncommon fatty acids from the membrane lipids of said seeds, yeasts and moulds by introducing, into the genome of said seeds, yeasts and moulds, a DNA sequence according to claim 1.

3. A method of accumulating uncommon fatty acids in the triacylglycerols of oil seeds, oleogenous yeasts and moulds comprising: removing said unclommon fatty acids from the membrane lipid of said seeds, yeasts and mou.lds, by introducing, into the genome of said seed, yeasts and moulds, a DNA sequence according to claim 1, together with a gene for an uncommon fatty acid such as medium chain, very long chain, hydroxy, epoxy and acetylenic acids.

4. A method according to claim 2. wherein said seeds, yeasts and moulds are transgeneic oil accumulating organisms engineered to produce an uncommon fatty acid, such as medium chain, long chain, hydroxy, epoxy and acetylenic acids.

5. A method according to claim 2, wherein said seeds, yeasts and moulds are crossed with transgenic oil accumulating organisms engineered to produce an uncommon fatty acid.

6. A method according to claim 2, wherein said phospholipase A₂ enzyme has specificity for octanoic (8:0), decanoic (10:0), and dodecanoic (12:0) acids

7. Transcenic oil accumulating organisms comprising, in their genome, a low molecular weight phospholipase A₂ gene having specificity for a particular uncommon fatty acid, and the gene for said uncommon fatty acid.

8 Transgenic organisms according to claim 7, which are selected from the group consisting of oil crops, yeasts, and moulds.

9. A method of obtaining oils, comprising accumulation of oils in organisms according to claim 7.

10. Oils obtainable by the method according to claim 9.
